(19) United States
(12) Patent Application Publication
(10) Pub. No.: US 2020/0143539 A1
(43) Pub. Date: May 7, 2020

(54) SPATIAL ANALYTICAL MICROBIAL IMAGING

(71) Applicant: Pei Zhang, Boston, MA (US)

(72) Inventor: Pei Zhang, Boston, MA (US)

(21) Appl. No.: 16/728,259

(22) Filed: Dec. 27, 2019

Related U.S. Application Data
(63) Continuation-in-part of application No. 15/183,768, filed on Jun. 15, 2016, now Pat. No. 10,550,416.
(60) Provisional application No. 62/175,514, filed on Jun. 15, 2015.

(51) Int. Cl. G06T 7/00; C12Q 1/6806

(52) U.S. Cl. CPC G06T 7/0012; C12Q 1/6806; G06T 2207/10056; G06T 2207/10028; G06T 2207/30024; G06T 2207/30242; G06T 2207/10064

(57) ABSTRACT

The present invention relates to microbial sample and standard preparation, staining and labeling, imaging and data acquisition as well as a method and apparatus for distinguishing objects of interest from other objects and background in an optical field. A microbial genomic analysis tool, Spatial Analytical Microbial Imaging (SAMI), provides the spatiotemporal and comparative intracellular ploidy, indicating the relative growth rate of cells in situ. Objects in a 2D or 3D optical field are tagged using fluorescence marker DNA binding to specifically visualize and semi-quantify the targeted objects in the sample, which allows them to be identified characterized and counted. In particular, inferential comparative genomic copy number (relative vitality), which is the relative amplification rate of cells related to the copy of genomic compounds in the cell comparing to those of the pure culture standards, are determined.

### FIELD OF THE INVENTION

The present invention relates to a method for analyzing a bacterial community using an inferential comparable method. In particular, the present invention relates to Spatial Analytical Microbial Imaging (“SAMI”).

### BACKGROUND

There is a lack of a generic tool in acquiring spatiotemporal and comparative genomic copy number in-situ for universal co-culture microbial community. Furthermore, many methods are limited to 2-dimensional (2D) observations. However, cells are in fact 3-dimensional (3D) structures and exist in 3D microbial consortia.

Some essential microbial cellular and community information are missing by most molecular fingerprinting techniques. For example, techniques such as Denaturing Gradient Gale Electrophoresis (DGGE)(Muyzer, Waal, & Uitierlinden, 1993)(US7560236B2), clone libraries (US20140228223A1), T-RFLP (Terminal Restriction Fragment Length Polymorphism) methods, flow cytometry, Comparative Genomic Hybridization (CGH) (Pinkel et al., 1998) (Francisco, Kallioniemi, Waldman, & Francisco, 2000), and DNA sequencing, real time-PCR (Pecoraro, Zerulla, Lange, & Soppa, 2011), need to destroy community structure and/or cellular integrity. As such, important information, such as spatial locus of cells and their in situ genomic copy number, is lost. Fluorescent In-situ hybridization (FISH) (as disclosed in, for example, U.S. Pat. Nos. 5,880,473 and 6,136,540) maintains the cellular integrity, but lacks the genomic copy number and demands major efforts in developing specific fluorescent markers, which allow for limited applications.

### SUMMARY OF THE DISCLOSURE

The present invention relates to microbial sample and standard preparation, staining and labeling, imaging and data acquisition as well as a computer-implemented method and apparatus for distinguishing objects of interest from other objects and background in an optical field. More particularly, in an embodiment a method of identifies, characterizes and counts objects in the 2D or 3D optical field which are tagged using fluorescence dye DNA binding to specifically visualize and semi-quantify the targeted objects in the sample. In particular, inferential comparative genomic copy number (relative vitality), which is the relative amplification rate of cells related to the copy of genomic compounds in the cell comparing to those of the pure culture standards, are determined.

In an embodiment, a method of spatial analytical microbial imaging is provided that includes growing a plurality of cultures, including a target microorganism culture and a slow growth microorganism culture, wherein cells of the slow growth microorganism culture have a lower genomic copy number than a genomic copy number of cells of the target microorganism culture; staining each of the plurality of cultures at an end of the time period with a fluorescent dye; imaging each of the plurality fluorescent stained cultures with a microscope and determining from the imaging an average fluorescent intensity for the targeted microorganism culture, a total fluorescent binding area for the targeted microorganism culture, and a total fluorescent binding area for the slow growth microorganism culture; comparing the total fluorescent binding area of the target microorganism culture to the total fluorescent binding area of the slow growth microorganism culture; and determining a genomic copy number or a relative vitality of the target microorganism culture.

In an embodiment, a method of spatial analytical microbial imaging is provided that includes growing a plurality of cultures, including a target microorganism culture, a slow growth microorganism culture, and a control microorganism culture, wherein cells of the slow growth microorganism culture have a lower genomic copy number than a genomic copy number of cells of the control microorganism culture, wherein each of the plurality of cultures are grown under a set of conditions, the set of conditions being substantially the same for the control microorganism culture and the target microorganism culture and including an effective time period, and the same effective duration; staining each of the plurality of cultures at an end of the time period with a fluorescent dye; imaging each of the plurality fluorescent stained cultures with a microscope and determining from the imaging an average fluorescent intensity for the targeted microorganism culture, a total fluorescent binding area for the targeted microorganism culture, an average fluorescent intensity for the control microorganism culture, and a total fluorescent binding area for the slow growth microorganism culture; comparing the average fluorescent intensity of the targeted microorganism culture to the average fluorescent intensity of the control microorganism culture and comparing the total fluorescent binding area of the targeted microorganism culture to the total fluorescent binding area of the slow growth microorganism culture; and determining a multi-dimensional location, a species, and a genomic copy number or a relative vitality of the targeted microorganism culture.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. For the purpose of illustrating the invention, the drawings show aspects of one or more embodiments of the invention. However, it should be understood that the present invention is not limited to the precise arrangements and instrumentalities shown in the drawings, wherein:

FIG. 1 is a graphic showing an exemplary use of Spatial Analytical Microbial Imaging (SAMI) to analyze a two species mixed community compare with pure culture standards in accordance with an embodiment of the present invention.

FIG. 2 shows an example in which pure culture standards (also referred to as “control” culture) and SAMI spatial distribution of the two species in (a), (b) The controls represent the true spatial distribution of the two species in the sample by utilizing autofluorescence emitted by the one species. HO and MA stain the nucleic acid of all species. In (c), (d), the spatial distribution of the two species via HO and MA nucleic acid staining using the SAMI method are shown.

FIG. 3 shows an example in which there is a 3D distribution of relative genomic copy number in the mixture of the two known species. In FIG. 3, blue circles represent the relatively faster growing cells, which have larger number of genomic copies than that of the growth standard; green circles represent equal growth cells with equal number of genomic copies as the growth standard; yellow circles represent the slower growth cells with less genomic copy than that of the growth standard. The growth standard can be the slow growing culture of oligoploid, merodiploid or polyploidy etc.

FIG. 4 depicts the percentage distribution of inferential comparative genomic copy number of a species in the mixed culture at different time phases as explained in 1.5 (below). The genomic copy number of each cell in the sample is evaluated by comparing the genome size with that of the growth standard with the genomic copy number of “1”. In FIG. 4, the black bars represent the cells with lower genomic copy number than that of the standard, indicating slower growing cells; the white bars represent the cells with equal genomic copy number as the standard, hence indicating equal growth rate; the doted bars represent the cells with higher genomic copy number than the standard, indicating faster growing cells. The time interval between each phase in this example is three days.

FIG. 5 shows the percentage distribution of inferential comparative genomic copy number of another species in the mixed culture at different time phases. The genomic copy number of each cell in the sample is evaluated by comparing the genome size with that of the growth standard with the genomic copy number of “1”. In FIG. 5, the black bars represent the cells with lower genomic copy number than that of the standard, indicating slower growing cells; the white bars represent the cells with equal genome copy number and growth rate as the standard; the doted bars represent the cells with higher genomic copy number than that of the standard, hence demonstrating a faster growth rate than the standard. The time interval between each phase in this example is three days.

FIG. 6 is a flowchart for spatial analytical microbial imaging in accordance with an embodiment of the present invention. The flowchart provides an overview of the process of using SAMI method in combination with SAMI software to obtain community spatiotemporal results. The system relationship among customer data input through software, database build up, and modeling is based on the mega-data from the database. The database and modeling results become resources for the customer to extract and utilize in evaluation, simulation, comparison or other data manipulation. The customer is able to extract data from the database through customized user interactive function and display in Visualization Engine (VE).

FIG. 7 is a flowchart for single time point spatial analytical microbial imaging data processing in accordance with an embodiment of the present invention. This chart outlines the processing steps for one time point data, and the road map set forth for the various processes, computations and manipulations of the acquired 2D and 3D data that is acted upon mainly through a custom data processor.

FIG. 8 is a flowchart for spatiotemporal spatial analytical microbial imaging data processing in accordance with an embodiment of the present invention. This flowchart explains the generation of the community temporal data based on single time point data. These results are saved through communication with the server and database for others to use and for modeling purposes. VE data can be displayed in multiple ways, such as in bar charts or a 5D diagraph. Customers can also utilize data from the database or modeling results through interactive customized end user function to compare/manipulate with previous data or other's data.

FIG. 9 depicts an overview of a technique for interactive data processing and utilization in accordance with an embodiment of the present invention. A communication road map between customers and the server is outlined. In this way, customers interact with the server to transfer and acquire data to share broader information in their data analysis. In the meantime, their data can also be saved in the server as part of the database for modeling and thus be available to be utilized by others. Modeling can be done based on customers' needs and design, such as by summarizing data according to their conditions and processing the best fit from the majority of data as a reference for community analysis.

### DESCRIPTION OF THE DISCLOSURE

The present invention overcomes the problems outlined above by determining the spatiotemporal and comparative intracellular ploidy that represents the growth rate of the cell. In an embodiment, allows cell structures to remain intact while identifying the genera of the cells in the mixed culture and their 2D or 3D locus and intercellular structure. The genomes of individual cells in the pure culture and the mixed culture are stained with fluorescent DNA markers and analyzed by microscopy and software. The average fluorescent intensity (AFI) and the total genomic fluorescent biding area (GFA) of pure cultures indicate the population AFI and GFA using inferential statistics. They are used as standards in comparison with the results of the sample to specify the genera, 2D or 3D locus and the relative vitality or a different category of each cell. The final results are presented in 2D or 3D. An embodiment provides a method for determining information of the relative genome size of the microorganisms in the mixture compare to those of their pure culture standards. Relative metabolic growth rate of the cells and the spatiotemporal change of the community are determined through inferential comparative genomic copy number evaluation. In this way, standards of pure cultures can be compared with the mixed cultures to evaluate the growth of the cells within the mixtures and gain multidimensional information of the microbial community.

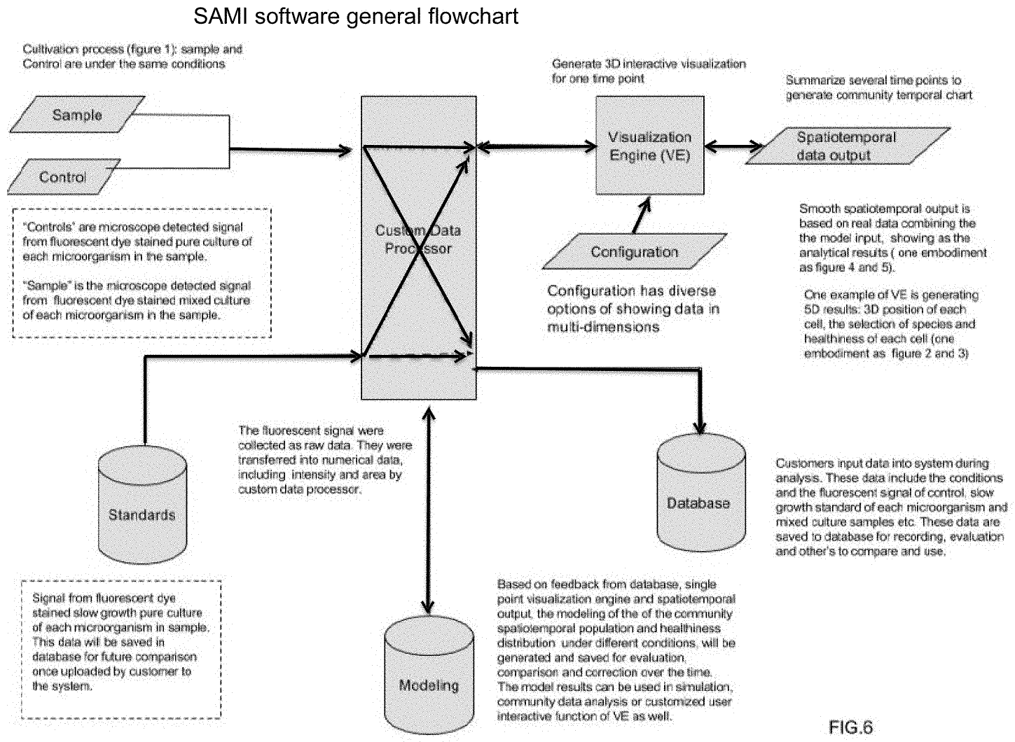

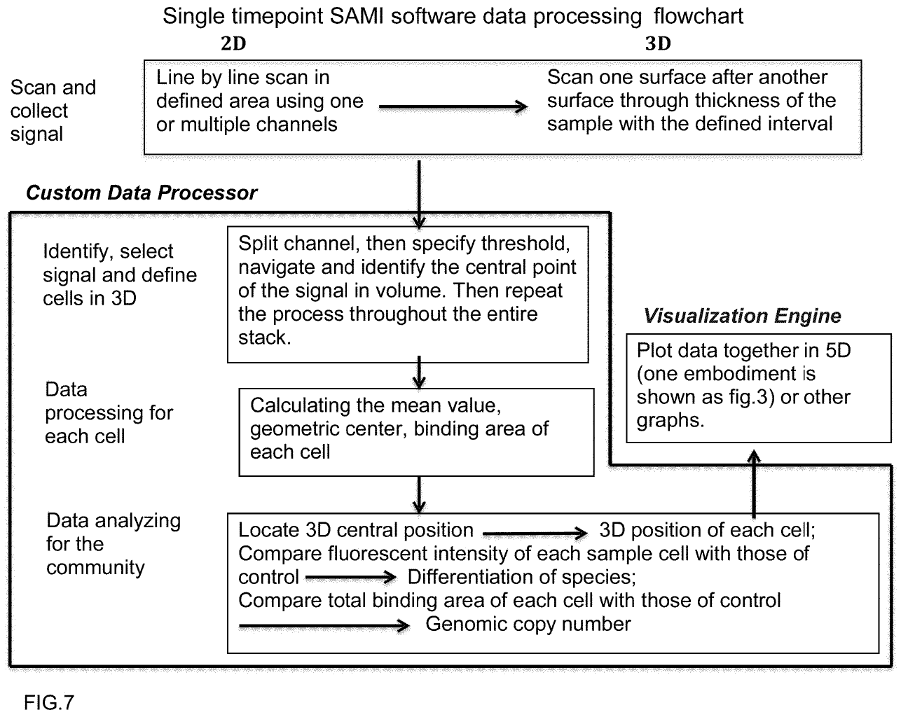

A flowchart is shown in FIG. 7. One example of the general system flowchart is shown in FIG. 6.

SAMI may be implemented in accordance with the following steps 1 to 4:

1 Standard and culture preparation:
Pure culture standard (i.e., the control microorganism culture) needs to be prepared under the same conditions and effective durations as the sample culture. In obtaining statistically valid data, sufficient number of cells need to be sampled in the pure culture standards in order to represent the population.

1.1 Pure culture growth standard

Pure culture growth standards (i.e., the slow growth microorganism culture) with any particular growth rates of the targeted microorganisms shall be prepared at corresponding specific conditions to maintain certain quantity of genomic copy number of each strain. In one embodiment, a slow growth microorganism culture is prepared under the condition of scarce nutrient to maintain low genomic copy number in the standards. Other methods, such as real time-PCR, fluorescence-activated cell sorting (FACS) analysis or radioactive labeling genome analysis can be used to quantify the exact copy numbers of the standards.

1.2 The targeted sample can be cultured or uncultured pure culture, single microbial species dominated microorganism culture, or mixed microorganism culture in suspension or solid material. If it is cultured microorganisms, then the pure culture (control microorganism culture) and the slow growth pure culture standards (slow growth microorganism culture) can be from different source. If it is uncultured microorganisms, the pure culture standards and the slow growth pure culture standards have to be isolated from the original flora but not necessarily to be sequenced or registered.

### 2. Staining and Labeling of the Sample.

### Fixation:

Pure culture (control microorganism culture) and slow growth pure culture standards (slow growth microorganism culture) can be fixed before to being evaluated as standards, or can be evaluated without first being fixed. The samples can also be fixed or processed without fixation. Fixed samples are more stable with higher accuracy, but unfixed samples can show the temporal information of the sample over time. In some embodiments, organic solvents are used to fix and permeabilize cells at the same time.

### Florescent Dye Staining:

Membrane permeable fluorescent dye binding with AT or GC base pair is used for signaling the corresponding DNA fragment and to semi-quantify the volume of the genome with 3D designation. Other particles or agents may be added to enhance florescence signal.

2.3 Imaging and data acquisition

### Microscope Preparation and Photographing

Samples and standards were visualized by Confocal Laser Scanning microscope (CLSM) or other type of microscope. The scan speed, pixel size and other parameters can be set for optimal performance. Imaging data are scanned through the third dimension, data is collected and transferred into a computer program, for example CLSM system software. A flowchart about the method is shown in FIG. 7.

3. Computer-implemented method for data analysis:

Images are analyzed in following procedure and logistics.

3.1 In both 2D and 3D data analysis, channels are split, thresholds are set to enclose most data point in a narrow range. The boundary of each cell is set. The values, such as mean value, geometric center, binding area, are analyzed based on the original imaging data acquired by LSM 510 or other software and the results were displayed. A flowchart about the method is shown in FIG. 7.

3.2 The same process is performed for the mixed samples, or the single cell dominant samples, the pure cultures, and the slow growth pure cultures.

3.3 The average fluorescence intensity (AFI), mean value, of each cell in mixed culture are compared to the mean value of the pure culture and the genera of each cell in the mixed culture image is identified.

4. Display of results:

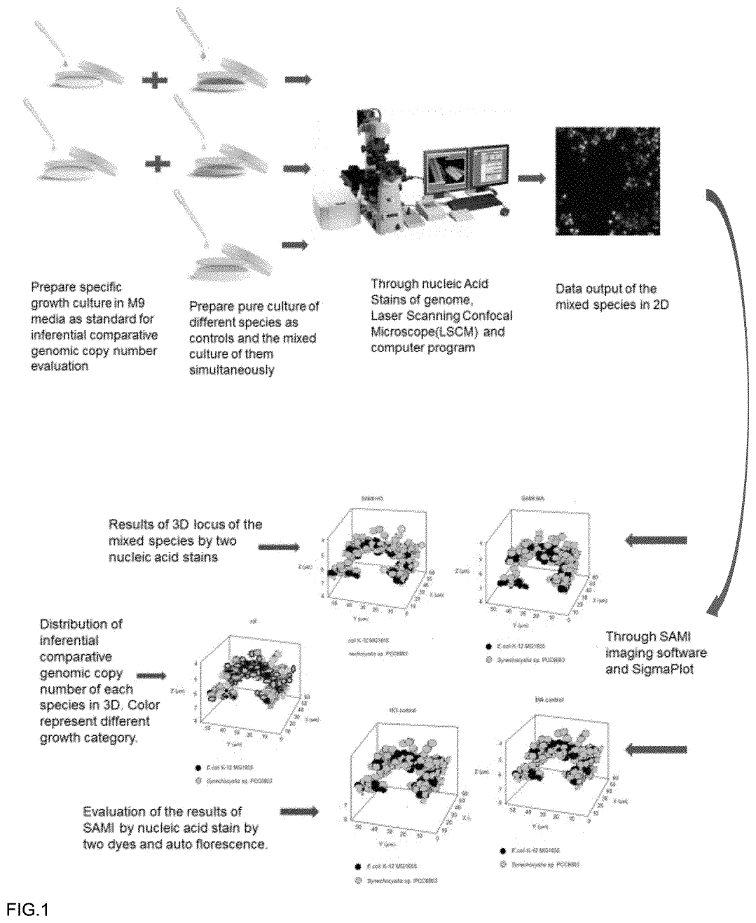

The identified results may be plotted using 3D data (x, y, z) in sigmaplot10.0 or other suitable software and compare with the control. The 3D distribution of the cells in an example are shown in FIG. 1. A flowchart about the method is shown in FIG. 7.

### Sample Preparation, Imaging and Analysis

1 Culture preparation

1.1 Pure culture standards preparation

Transfer a single colony of each pure culture into separate nutrient medium then incubate them at the desirable conditions. This solution serves as pure culture standard solution. After being preserved in a refrigerator, it may be considered to be in the same condition as the time point before preservation in the refrigerator. In other words, the preservation time is excluded from the effective duration.

1.2 Mixed culture sample preparation

Mixture of the pure cultures is cultivated by mixing the pure culture solutions as explained at 1.1 (above) together under certain conditions and each of the pure culture is cultivated again at the same conditions as the mixed culture to ensure they grow under the same conditions with the same effective duration. After being preserved in a refrigerator, it may be considered to be under the same condition as the time point before preservation in the refrigerator. In other words, the preservation time is excluded from the effective duration.

1.3 Specific growth culture standard (slow growth microorganism culture).

In one embodiment, 1 ml of the pure culture solution, explained at 1.1, was transferred into a specific growth medium and cultivated for certain time under certain condition to maintain stable growth rate. The process was repeated multiple times. The genomic copy number of the specific growth culture corresponding to the specific species and culture conditions does not necessarily have to be determined depend on different implementations. In some embodiments, the growth condition is used as criteria and/or relative growth rate compare to the growth standard is sufficient to evaluate the growth, then a relative growth rate (a relative vitality) will be used, therefore no need to invest the absolute genomic copy number. However, alternatively, if the genomic copy number of the growth standard is important, then genomic copy number of the specific growth culture can be determined by real time-PCR and fluorescence-activated cell sorting (FACS) analysis or radioactive labeling genome analysis or other methods.

2. Sample fixation, stain and preparation for imaging

### 2.1 Fixation

In an embodiment, samples were washed twice with buffer before fixation. In another embodiment, the fixation time used for mixed bacteria samples was 3 hours. In one embodiment, the glass slides with samples on it were transported on a sterilized petri-dish in a biological hood and immersed in 1 ml of 4% buffered paraformaldehyde solution for 3 hours to fix the cells following by air drying. The buffered solution was made from stock paraformaldehyde (16% paraformaldehyde, CAS #30525-89-4, Electron Microscopy Sciences) in 2M NaCl and 0.1M PBS buffer (15 mM MgCl2; 0.8 g sodium chloride, 0.2 g potassium chloride, 1000 ml sterile distilled water, pH 7.5). In the hood, the sample was covered in petri-dish at room temperature for 3 hours. In one embodiment, organic solvent is used to fix and permeabilize the cells.

2.2 Fluorescent dye staining:

Fluorescent dye staining can be performed with membrane permeable dye binding to AT or GC base pair. In one embodiment, BisBenzimide H 33258(HO) is used. It is membrane-permeable and intercalates in A-T regions of DNA. An aliquot of 10 mg/ml Bisbenzimide H 33258, HO Solutions (DNA Quantitation Kit, DNA-Q, Sigma) was first diluted 10-fold with molecular biology grade water (W 4502, Sigma) to a concentration of 1 mg/ml. Another membrane-permeable dye be used in one embodiment is Mithramycin A (MA) (M6891Mithramycin A, Sigma) which is a natural polycyclic aromatic polyketide produced by various Streptomyces species, that preferentially binds to GC-rich sequences in DNA. In one embodiment, Mithramycin A solution was dissolved in methanol (MX0485-7 EMD) to form a 10 mg/ml solution. This solution was diluted 10 fold with 300 mM MgCl2 to form a 1 mg/ml stock solution. The working solution was made fresh every time by mixing MA stock solution, HO stock solution, 10× Fluorescent Assay Buffer (F 7171, Sigma) as well as 20×MgCl2 and Molecular Biology Grade Water (W 4502, Sigma). The final concentration of MgCl2 was 15 mM at the time of measurement. The dye solution was set under dark conditions at room temperature for 20 minutes to reach equilibrium. Then 1 ml of the mixed dye working solution was added to each sample for 20 minutes. In one embodiment, samples were washed once with buffer before staining. In one embodiment, nanoparticles, such as resonant dielectric nanoparticles are added into the dye solution to enhance fluorescence signal. In another embodiment, nanoparticles, such as dielectric nanoantennas, are added into dye solution to improve fluorescence signal.

3. Microscope preparation and imaging

For visible light and high numerical aperture objectives (>0.8) a pixel size of ˜0.1-0.2 μm is recommended. In one embodiment, the pixel size of the CLSM system was optimized to 0.116 μm. In order to process fast dynamic scans, a sequential raster scan was used and the scan speed was optimized at 0.9 μs/pixel to reduce delays between acquisitions. The other parameters were optimized and set up accordingly by using LSM 510 or other software. In one embodiment, these parameters were as follows: Amplifier offset at 0.1; Amplifier gain at 1; Power of 405 nm at 0.5 mW; Pinhole at 0.58 Airy equivalent; Optical slice 0.6 μm; Frame size at 512 μm×512 μm; Interval at 0.1 μm.

4. Image analysis using software 2D and 3D functions:

For both 2D and 3D data analysis, channels were split; thresholds were set to enclose most data point in a narrow range. The boundary of each cell is set. The values, such as mean value, geometric center, binding area, were analyzed based on the original imaging data acquired by CLSM or other software and the results were displayed.

The same processes are done to the said mixed samples, single cell dominated samples, pure cultures, and the slow growth pure cultures.

Comparison of the average fluorescence intensity (AFI), mean value, of each cell in mixed culture to the mean value of the pure culture and identify the genera of each cell in the mixed culture image. For mixed culture sample, AFI of each pure culture of the mixed species is pre-evaluated first. If they are well separated, then SAMI is applicable; if not, then SAMI is not applicable.

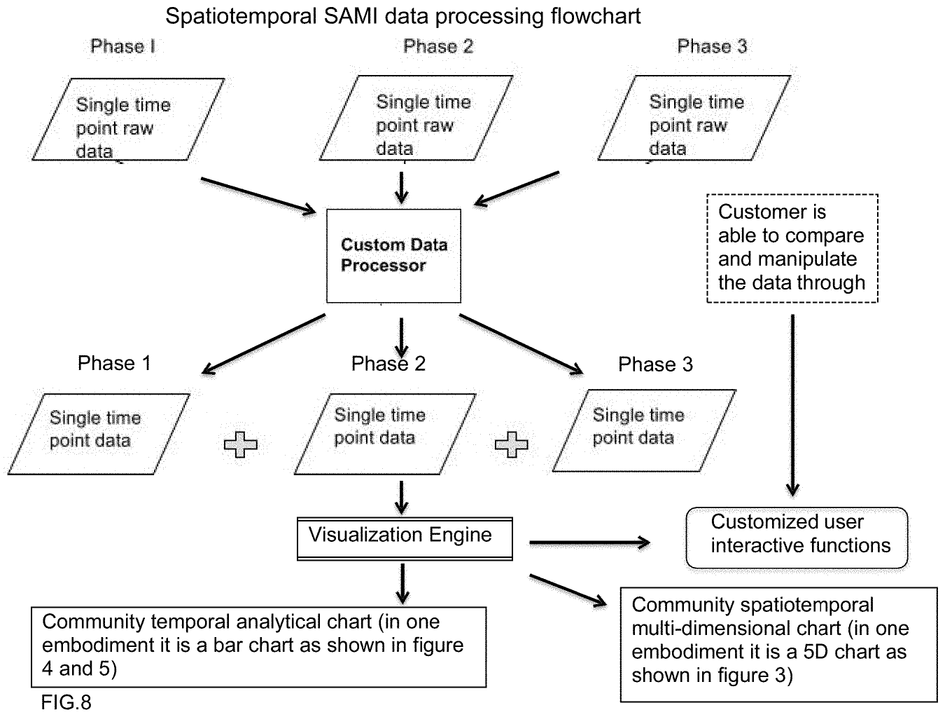

Comparison of the total genomic fluorescent binding area (GFA) of the cells in the said mixed culture to the average value of the said slow growth pure culture standard of the same species. Calculate the genomic copy number or evaluate the relative vitality of each cell of the said mixed culture sample compare to the said slow growth pure culture and plot the data in 2D or 3D. When evaluating the relative vitality, the slow growth pure culture genomic copy number is considered as one, then the GFA of each cell is compared to that of the slow growth pure culture, from which it can be determined whether the relative vitality of each cell is equal to one, higher than one, or lower than one. Alternatively, the relative vitality of each cell is determined by calculating that the number of times the GFA of each cell is to the GFA of the slow growth pure culture. The results then may be displayed in multi-dimensions. A flowchart of a single time point data processing is shown in FIG. 7. The data processing can be used to display single time point community distribution or in community temporal results in VE as shown in FIG. 8.

### Example 1

1 Culture preparation

1.1 To prevent contamination. E. coli K-12 MG1655 was transformed by inserting ampicillin resistant genes (AmpR) through electroporation. The size of the ampicillin resistant gene was approximately 1.25 kb, which is not significant in comparison to the whole genome size of E. coli K-12 MG 1655.

1.1.1 Pipette 40 μl of electro-competent cells (E. coli K-12 MG 1655) into ice-cold sterile 0.5 ml microfuge tubes. Place the cells on ice, as well as electroporation cuvettes.

1.1.2 Add 10 ng to 25 ng of DNA plasmid (1-2 μl) to each microfuge tubes and incubate in ice for 30 to 60 seconds, including controls.

1.1.3 Set the electroporation apparatus to electrical pulse of 25 μF capacitance, 2.5 kV, and 2000 resistance.

1.1.4 Pipette the DNA/cell mixture into the cold cuvette, tap the solution to ensure bacteria cell touch the bottom of the cuvette. Dry outside of the cuvette, put it into the device then push the pulse bottom.

1.1.5 Remove the cuvette and add 1 ml of LB media at room temperature as soon as possible.

1.1.6 Transfer the cells to a 17×100 mm polypropylene test tube, incubate under 37° C. for 1 hour.

1.1.7 Plate 50 μl, 100 μl, 150 μl, 200 μl of the electroporation cells onto the LB agar medium place containing 20 mM MgSO4, and 200 μg/ml ampicillin.

1.1.8 Wait until the liquid is absorbed and then invert the plate and incubate in 37° C. for 12-16 hours.

1.2 E. coli K-12 MG 1655 pure culture

1.2.1 Transfer a single colony of E. coli K-12 MG 1655 from an LB agar plate into a 50 ml sterilized polypropylene tube containing 25 ml LB solution supplemented with 25 μl of 200 μg/μl ampicillin sodium salt (69523, Sigma).

1.2.2 Incubate the tube in an incubator shaker (Fisher scientific isotemp. E-class incubator) at 225 rpm and 37° C. for 12 hours.

1.3 E. coli K-12 MG 1655 pure culture standard

1.3.1, One ml of E. coli K-12 MG 1655 culture was transferred from the incubator shaker to an alcohol sterilized glass slide, then placed in a petri-dish and kept at room temperature (27° C.±1° C.).

1.3.2 Two ml of freshly made BG-11 media was added daily onto the glass slide for three days to support E. coli K-12 MG1655 pure culture growth on the glass surface, which was kept under the same conditions as the mixed culture. After being preserved in a refrigerator, it may be considered at the same condition as the time point before preservation in the refrigerator; i.e., the preservation time is excluded from the effective duration.

1.4 Synechocystis sp. PCC 6803 pure culture

1.4.1 One colony of Synechocystis sp. PCC 6803 from a BG-11 agar plate was transferred into 25 ml BG-11 media in a 50 ml sterilized polypropylene tube in a UV sterilized biological hood.

1.4.2 The tube was set under laboratory room light at room temperature (27° C.±1° C.) to allow Synechocystis Sp. PCC6803 to grow in suspension.

1.4.3 Synechocystis sp. PCC 6803 pure culture standard

1.4.4 One ml of suspended Synechocystis sp. PCC 6803 culture was transferred onto an alcohol sterilized glass slide (as explained in E. coli pure culture) in a UV sterilized biological hood.

1.4.5 Two ml of BG-11 media was added on the glass slide every day for three days to allow the growth of the Synechocystis sp. PCC 6803 pure culture on the glass surface. After being preserved in a refrigerator, it may be considered at the same condition as the time point before preservation in the refrigerator. The preservation time is excluded from the effective duration.

1.4.6 Mixed culture preparation

E. coli K-12 MG1655 was first cultivated on a glass slide surface for three days as described earlier as for the pure culture control. Then 1 ml of pure culture Synechocystis sp. PCC 6803 suspension was added onto the E. coli K-12 MG1655 glass slide located in the sterile petri-dish to allow the growth of both bacterial species as phase I. 2 ml of BG-11 media was then added to the glass slide every day for three days as phase 2 and the mixture was allowed to grow at room temperature (27° ° C.±1° C.) for another three days as phase 3. Pure culture controls of Synechocystis sp. and E. coli K-12 MG1655, were prepared concurrently.

1.4.7 E. coli K-12 MG1655 slow growth culture standard.

Firstly a single colony of E. coli K-12 MG1655 was carefully isolated from the LB agar plate and transferred to 10 ml M9 media. The culture was then incubated in a shaker (Fisher scientific isotemp. E-class incubator) at 225 rpm, 27° C.±1° C. for 3 days. Ten 1 ml of the solution were then transferred into 10 ml of fresh M9 media and grown under the same conditions for 3 more days. This process was repeated a minimum of 3 times in order to obtain a slow growth culture standard. After being preserved in a refrigerator, it may be considered at the same condition as the time point before preservation in the refrigerator. The preservation time is excluded from the effective duration.

1.4.8 Synechocystis sp. PCC 6803 slow growth culture standard

It has been previously reported that Synechocystis sp. PCC 6803 motile wild type has about 60 genomes per cell in stationary phase and this number varies for different strains. The genome of Synechocystis sp. PCC 6803 slow growth pure culture was first investigated and inferential statistics were used in comparative genomic analysis to obtain comparative genomic copy number of each Synechocystis sp. PCC 6803 in the mixture sample.

A single colony of Synechocystis sp. PCC 6803 was transferred from an LB agar plate to 10 ml of BG-11 media. The culture grew for 3 days under room light and room temperature (27° C.±1° C.). 1 ml of it were then transferred into 10 ml of fresh BG-11 media and allowed to grow for 3 more days. The process was repeated 3 times. After being preserved in a refrigerator, it is may be considered at the same condition as the time point before preservation in the refrigerator. The preservation time is excluded from the effective duration.

Inferential statistical analysis was used in obtaining genomic copy number standard of the slow growth cells. The absolute genomic copy number of the slow growth pure culture can be quantified by real-time PCR, a spectroscopic method, fluorescence-activated cell sorting analysis, radioactive labeling genome analysis or other quantitative method. When evaluating the relative genomic copy number (relative vitality), the slow growth pure culture genomic copy number is considered as one, then the GFA of each cell is compared to that of the slow growth pure culture, from which it can be determined whether the relative vitality of each cell is equal to one, higher than one, or lower than one. Alternatively, the relative vitality of each cell is determined by calculating the number of times the GFA of each cell is to the GFA of the slow growth pure culture. The results then may be displayed in multi-dimensions. A flowchart of a single time point data processing is shown in FIG. 7. The data processing can be used to display single time point community distribution or in community temporal results in VE as shown in FIG. 8.

2. Sample fixation, stain and preparation for imaging

### 2.1 Fixation

Because the nucleic acid dyes utilized in this study were membrane-permeable, samples could be fixed for one point sampling or processed without fixation for sequential sampling. The temporal observation of samples was performed without fixation. When fixation was utilized, samples were washed twice with buffer and then fixed for 3 hours in glass slides. The glass slides were then transported on a sterilized petri-dish to a biological hood and immersed in 1 ml of 4% buffered paraformaldehyde solution for 3 hours at room temperature. The buffered solution was made from stock paraformaldehyde (16% paraformaldehyde, CAS #30525-89-4, Electron Microscopy Sciences) in 2M NaCl and 0.1M PBS buffer (15 mM MgCl2; 0.8 g sodium chloride, 0.2 g potassium chloride, 1000 ml sterile distilled water, pH 7.5). In one embodiment, Saponin is used as organic solvent to fix and permeabilize the cells.

2.2 Fluorescent dye staining

Fluorescent dye staining was performed with BisBenzimide H 33258(HO) which is membrane-permeable andintercalates in A-T regions of DNA. An aliquot of 10 mg/ml Bisbenzimide H 33258, HO Solutions (DNA Quantitation Kit, DNA-Q, Sigma) was first diluted 10-fold with molecular biology grade water (W 4502, Sigma) to a concentration of 1 mg/ml. Another membrane-permeable dye utilized in this study was Mithramycin A (MA) (M6891Mithramycin A, Sigma) which is a natural polycyclic aromatic polyketide produced by various Streptomyces species, that preferentially binds to GC-rich sequences in DNA. Mithramycin A solution was dissolved in methanol (MX0485-7 EMD) to form a 10 mg/ml solution. This solution was diluted 10-fold with 300 mM MgCl2 to form a 1 mg/ml stock solution. The working solution was made fresh every time by mixing MA stock solution, HO stock solution, 10× Fluorescent Assay Buffer (F 7171, Sigma) as well as 20×MgCl2 and Molecular Biology Grade Water (W 4502, Sigma). The final concentration of MgCl2 was 15 mM at the time of measurement. The dye solution was set under dark conditions at room temperature for 20 minutes to reach equilibrium. Then 1 ml of the mixed dye working solution was added to each sample for 20 minutes. The quantification of DNA has been reported to require a high salt concentration. Higher salt concentrations appear to cause the dissociation of proteins from DNA, allowing better binding of the dye molecules with AT or GC base pairs in minor groove of DNA. For peak fluorescence, at least 200 mM NaCl is required for purified DNA and 2.0 to 3.0 M for crude samples. Mg2+ ions have no effect on the assay in the final concentration range from 0.5 mM to 0.1 M and the salt concentrations of 3 M NaCl will not affect the assay. Samples were washed once with buffer before staining. The two dyes were combined and stained once for all the samples. The composition of mixed dye of HO and MA is shown in Table 1.

TABLE 1
Components of mixed dye of HO and M/X

| Dyes | HO | MA |
|---|---|---|
| 1 mg/ml dye | 5 μl | 5 μl |
| 20*MgCl₂ | — | 0.16 ml |
| 10* buffer (5 mM Tris-HCl, | 0.5 ml | 0.5 ml |
| pH 7.6, 8 mM NaCl) | | |

| | |
|---|---|
| Sterile water | 3.83 ml |
| Total | 5 ml |

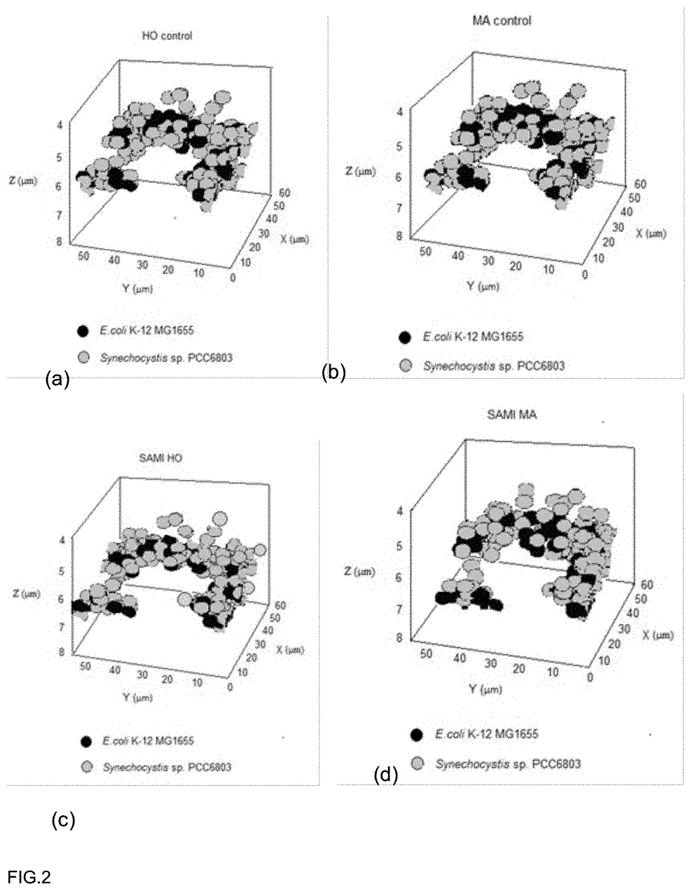

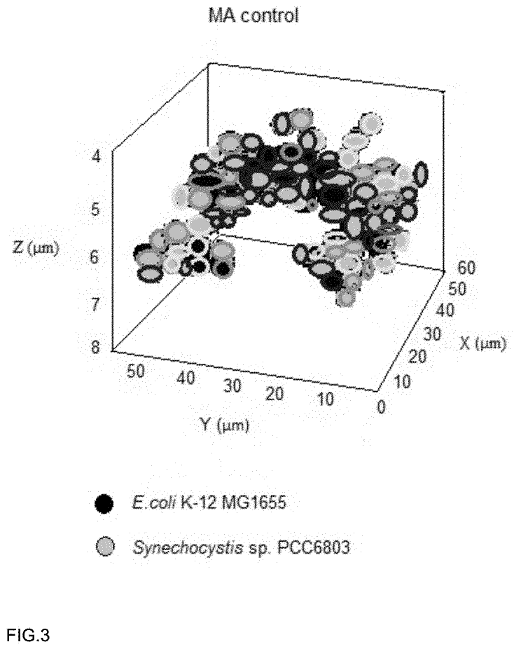

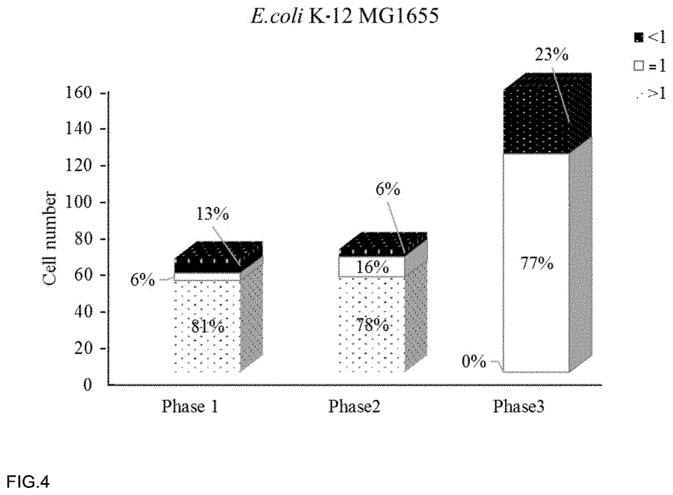

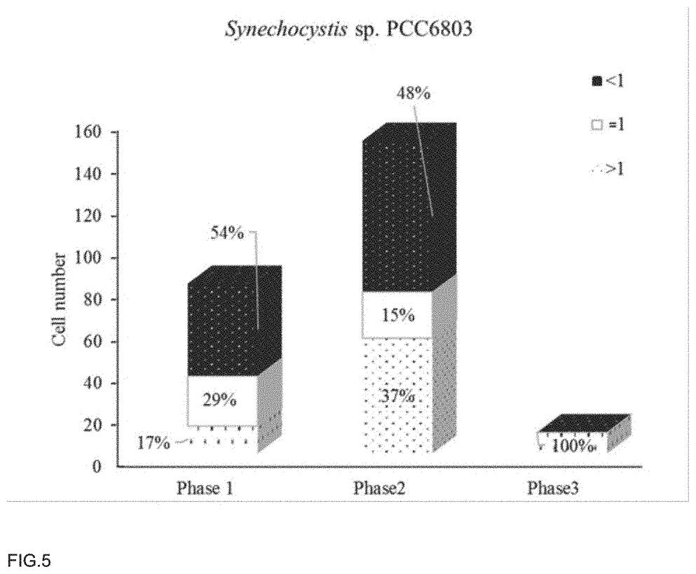

Results are shown in FIGS. 2 to 5. In one embodiment, resonant dielectric nanoparticles, such as Subquarter Micrometer Silicon Spheres, are added into the dye solution to enhance fluorescence signal. In another embodiment, dielectric nanoantennas, such as all-silicon nanoantennas, are added into dye solution to improve fluorescence signal.

3. Microscope preparation and imaging

For visible light and high numerical aperture objectives (>0.8) a pixel size of ˜0.1-0.2 μm is recommended. In one embodiment, the pixel size of the CLSM system was optimized to 0.116 μm. In order to process fast dynamic scans, Sequential raster scan was used and the scan speed was optimized at 0.9 μs/pixel to reduce delays between acquisitions. The other parameters were optimized and set up accordingly by using LSM 510 software or other software program. In one embodiment, these parameters were as follows: Amplifier offset at 0.1; Amplifier gain at 1; Power of 405 nm at 0.5 mW; Pinhole at 0.58 Airy equivalent; Optical slice 0.6 μm; Frame size at 512 μm×512 μm; Interval at 0.1 μm.

4. Image analysis using software 2D and 3D functions:

Both 2D and 3D data analysis, channels were split; thresholds were set to enclose most data point in a narrow range. The boundary of each cell is set. The values, such as mean value, geometric center, binding area, were analyzed based on the original imaging data acquired by CLSM software or other software program and the results were displayed.

The same processes are done to the said mixed samples and pure cultures and the slow growth pure cultures.

For the mixed culture of Synechocystis sp. PCC 6803 and E. coli K-12 MG1655, AFI of each pure culture was pre-evaluated first under the same condition. The value showed a well separation of the two species, so SAMI is applicable to the mixed sample of Synechocystis sp. PCC 6803 and E. coli K-12 MG1655.

Then the average fluorescence intensity (AFI), mean value, of each cell in mixed culture is compared to the mean value of the pure culture and identify the genera of each cell in the mixed culture image.

Comparison of the total genomic fluorescent binding area (GFA) of the cells in the said mixed culture to the average value of the said slow growth pure culture standard of the same species. Calculate the genomic copy number or evaluate the relative vitality of each cell of the said mixed culture sample compared to the said slow growth pure culture and plot the data in 2D or 3D. When evaluating the relative vitality, the slow growth pure culture genomic copy number is considered as one, then the GFA of each cell is compared to that of the slow growth pure culture, from which it can be determined whether the relative vitality of each cell is equal to one, higher than one, or lower than one. Alternatively, the relative vitality of each cell is determined by calculating the number of times the GFA of each cell is to the GFA of the slow growth pure culture.

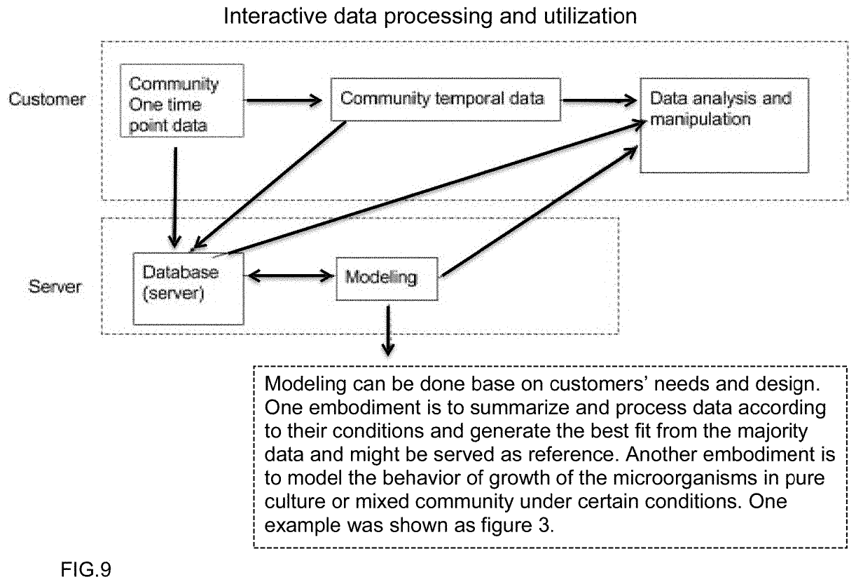

Results are shown in FIGS. 2-5. The flowcharts about the system are shown in FIG. 6-9.

A method for analyzing a bacterial community comprising schematic culture preparation of the targeting species under a controlled conditions and timing. Fixation or without fixation, then said culture be simultaneously stained with the specific fluorescent dyes.

Imaging and data acquisition of the said fluorescent stained culture by Confocal Laser Scanning Microscope (CLSM) or other microscope using specific single channel or multi-channel/multi-track scanning program with the optimal scan speed, pixel size and other parameters in three dimension as shown in FIG. 7.

Post data processing of the said imaging data by the specific imaging analysis software program functions, system architecture and the road map of the various processes, computations, database, modeling and manipulations of information are shown in FIG. 6 to 9. These data can be used to display single point time community distribution or in community temporal results in VE as shown in FIG. 8.

After processing with the said specific imaging software program functions, the analytical data of each cell were plotted in 2D or 3D by the specific software.

The method as above, wherein the said schematic culture preparation includes preparation of the pure culture, the slow growth pure culture standard of each species and the mixed culture of all the said species.

The method as above, wherein the specific timing of the cultivation refers to first prepare the slow growth pure culture to the stationary phase, then cultivate the said pure culture and the said mixed culture with the same effective duration. The effective time periods and the effective duration for the control microorganism culture, the slow growth microorganism culture, and the target microorganism culture exclude any time the respective cultures are preserved.

The method as above, wherein the said controlled conditions refer to the concentration and the composition of the growth media, the frequency of feeding the microorganisms, the light intensity and the incubation temperature corresponding to specific growth rate of the culture.

The method as above, wherein the said slow growth pure culture standard will be used as a standard to evaluate the genomic copy number of the cells in the said mixed culture.

The method as above, wherein the said slow growth pure culture standard can be quantified for the absolute genomic copy number by using real time-PCR or fluorescence-activated cell sorting (FACS) analysis or radioactive labeling genome analysis. In another embodiment, the said slow growth pure culture will be used as standard without quantifying the copy number of genome.

The method as above, wherein the said fluorescent binding dyes have binding mechanisms that allow them to bind or partially bind to the genome of the cell of the said culture and signaling proportionally to the amount of the DNA base pair of the cells of the said culture.

The method as above, wherein the said culture is fixed in one embodiment for better resolution of signals. Wherein in another embodiment, the culture is not fixed, then a membrane permeable said fluorescent binding dye is used to stain the said culture in getting temporal data.

The method as above, wherein the said pure cultures need to be pre-evaluated to validate SAMI is applicable by the following a) to f):
a) Grow pure culture of interested species under the said controlled conditions;
b) Said fixing or without fixing the culture;
c) Stain the said culture with the said specific fluorescent DNA binding dyes.
d) Said Imaging and data acquisition by CLSM
e) Said data processing and analyzing using specific imaging software functions.
f) Evaluate the results, if there is no significant difference among the species then SAMI is not applicable to the samples; if there is a significant difference among the species then SAMI is applicable to the samples.

The method as above, wherein the said imaging analysis software functions compose 2D and 3D data analysis, channels were split; thresholds were set to enclose most data point in a narrow range. The boundary of each cell is set. The values, such as mean value, geometric center, binding area, were analyzed based on the original imaging data acquired by CLSM or other software and the results were displayed.

The same processes are done to the said mixed samples and or the pure cultures and the slow growth pure cultures.

Comparison of the average fluorescence intensity (AFI), mean value, of each cell in mixed culture to the mean value of the pure culture and identify the genera of each cell in the mixed culture image.

Comparison of the total genomic fluorescent binding area (GFA) of the cells in the said mixed culture to the average value of the said slow growth pure culture standard of the same species. Calculate the genomic copy number or evaluate the relative vitality of each cell of the said mixed culture sample compare to the said slow growth pure culture and plot the data in 2D or 3D. When evaluating the relative vitality, the slow growth pure culture genomic copy number is considered as one, then the GFA of each cell is compared to that of the slow growth pure culture, from which it can be determined whether the relative vitality of each cell is equal to one, higher than one, or lower than one. Alternatively, the relative vitality of each cell is determined by calculating how many times the GFA of each cell is to the GFA of the slow growth pure culture.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

What is claimed is:

1) A method of spatial analytical microbial imaging comprising:
growing a plurality of cultures, including a target microorganism culture and a slow growth microorganism culture, wherein cells of the slow growth microorganism culture have a lower genomic copy number than a genomic copy number of cells of the target microorganism culture;
staining each of the plurality of cultures at an end of the time period with a fluorescent dye;
imaging each of the plurality fluorescent stained cultures with a microscope and determining from the imaging an average fluorescent intensity for the targeted microorganism culture, a total fluorescent binding area for the targeted microorganism culture, and a total fluorescent binding area for the slow growth microorganism culture;
comparing the total fluorescent binding area of the target microorganism culture to the total fluorescent binding area of the slow growth microorganism culture; and
determining a genomic copy number or a relative vitality of the target microorganism culture.

2) The method of spatial analytical microbial imaging of claim 1, wherein the target microorganism culture is a known single microbial species dominated microorganism culture or a pure microorganism culture.

3) The method of spatial analytical microbial imaging of claim 1, wherein the slow growth microorganism culture is a culture grown from a single microbial species, wherein the single microbial species is the same as the dominant microbial species in the target microorganism culture or a microbial species in the target pure microorganism culture.

4) The method of spatial analytical microbial imaging of claim 1, wherein the staining step includes using organic solvent for fixing and permeabilization and adding nano or micro scale particles or nano or micro scale structures to enhance florescence signal.

5) The method of spatial analytical microbial imaging of claim 1, wherein the slow growth microorganism culture is quantified for an absolute genomic copy number using real time-PCR, fluorescence-activated cell sorting analysis, or radioactive labeling genome analysis or other quantitative method.

6) The method of spatial analytical microbial imaging of claim 1, wherein the slow growth culture is a standard against which the genomic copy number or relative vitality of cells in the target microorganism culture is evaluated, further including comparing a total genomic fluorescent binding area of each cell in the target culture to an average value of total genomic fluorescent biding area of the slow growth microorganism culture, wherein a genomic copy number is set as one unit in order to facilitate comparisons of the relative vitality of each cell to the slow growth microorganism culture.

7) The method of spatial analytical microbial imaging of claim 1, further including determining a genomic copy number or relative vitality of each cell of the target culture, comparing to the slow growth microorganism culture, recording results, composing more than 2-dimensional and 3-dimensional data analysis, splitting channels, setting thresholds, setting boundary of each cell, calculating values based on imaging data acquired by the microscope, and displaying results.

8) The method of spatial analytical microbial imaging of claim 1, further including, prior to the step of growing the plurality of cultures, growing the slow growth microorganism culture in a medium designed for slow growth microorganism culture for more than three days, transferring a volume of the slow growth microorganism culture into a larger volume of the slow growth medium, growing the slow growth microorganism culture for more than three days, repeating the growing, transferring, and growing steps at least three times.

9) The method of spatial analytical microbial imaging of claim 8, further including preserving the slow growth microorganism culture is preserved in a refrigerator, wherein after the slow growth microorganism is preserved in the refrigerator, the slow growth microorganism culture is considered at the same condition as before preservation in the refrigerator.

10) A method of spatial analytical microbial imaging comprising:
growing a plurality of cultures, including a target microorganism culture, a slow growth microorganism culture, and a control microorganism culture, wherein cells of the slow growth microorganism culture have a lower genomic copy number than a genomic copy number of cells of the control microorganism culture, wherein each of the plurality of cultures are grown under a set of conditions, the set of conditions being substantially the same for the control microorganism culture and the target microorganism culture and including an effective time period, and the same effective duration;
staining each of the plurality of cultures at an end of the time period with a fluorescent dye;
imaging each of the plurality fluorescent stained cultures with a microscope and determining from the imaging an average fluorescent intensity for the targeted microorganism culture, a total fluorescent binding area for the targeted microorganism culture, an average fluorescent intensity for the control microorganism culture, and a total fluorescent binding area for the slow growth microorganism culture;
comparing the average fluorescent intensity of the targeted microorganism culture to the average fluorescent intensity of the control microorganism culture and comparing the total fluorescent binding area of the targeted microorganism culture to the total fluorescent binding area of the slow growth microorganism culture; and
determining a multi-dimensional location, a species, and a genomic copy number or a relative vitality of the targeted microorganism culture.

11) The method of spatial analytical microbial imaging of claim 10, further including repeating the growing, staining, imaging, and comparing steps for each of the plurality of cultures for a plurality of growth time periods, comparing and integrating the data and displaying results from the plurality of growth time periods on the multi-dimensional graph.

12) The method of spatial analytical microbial imaging of claim 10, wherein the target microorganism culture is a mix of multiple species of microorganisms.

13) The method of spatial analytical microbial imaging of claim 12, wherein one target species in the mix of multiple species of microorganisms corresponds to one slow growth microorganism culture of the same species and one control microorganism culture of the same species.

14) The method of spatial analytical microbial imaging of claim 10, wherein the set of conditions includes a composition of the growth media, a concentration of each composition, a frequency of feeding the microorganisms, a light intensity, an incubation temperature, and an effective duration of incubation time.

15) The method of spatial analytical microbial imaging of claim 10, wherein the effective time periods and the effective duration for the control microorganism culture, the slow growth microorganism culture and the target microorganism culture exclude any time the respective cultures are preserved.

16) The method of spatial analytical microbial imaging of claim 10, wherein the staining step includes using organic solvent for fixing and permeabilization and adding nano or micro scale particles or nano or micro scale structures to enhance florescence signal.

17) The method of spatial analytical microbial imaging of claim 10, wherein the slow growth microorganism culture is quantified for an absolute genomic copy number using real time-PCR, fluorescence-activated cell sorting analysis or radioactive labeling genome analysis or other quantitative methods.

18) The method of spatial analytical microbial imaging of claim 10, further including determining an average fluorescence intensity of each cell of the control microorganism culture and determining whether there is a significant difference between the average fluorescence intensity of the control microorganism cultures.

19) The method of spatial analytical microbial imaging of claim 18, further including, when the difference between the average fluorescence intensity of cells in the control microorganism cultures is significant, comparing an average fluorescence intensity of each cell in the target microorganism culture to the average fluorescence intensity of each of the control microorganism culture, and identifying a genera of each cell in a multi-dimensional image of the target culture.

20) The method of spatial analytical microbial imaging of claim 10, wherein the slow growth culture is a standard against which the genomic copy number or relative vitality of cells in the target microorganism culture is evaluated, further including comparing a total genomic fluorescent binding area of each cell in the target culture to an average value of total genomic fluorescent biding area of the slow growth microorganism culture, wherein a genomic copy number is set as one unit in order to facilitate comparisons of the relative vitality of each cell to the slow growth microorganism culture.

21) The method of spatial analytical microbial imaging of claim 10, further including determining a genomic copy number or relative vitality of each cell of the target culture, comparing to the slow growth microorganism culture, plotting a location of each cell, a microorganism identification, and a genomic copy number or a relative vitality of each cell, composing more than 2-dimensional and 3-dimensional data analysis, splitting channels, setting thresholds, setting boundary of each cell, calculating values based on imaging data acquired by the microscope, and displaying results in multi-dimensions.

22) The method of spatial analytical microbial imaging of claim 10, further including, prior to the step of growing the plurality of cultures, growing the slow growth microorganism culture in a medium designed for slow growth microorganism culture for more than three days, transferring a volume of the slow growth microorganism culture into a larger volume of the slow growth medium, growing the slow growth microorganism culture for more than three days, repeating the growing, transferring, and growing steps at least three times.
